(19) United States
(12) Patent Application Publication
(10) Pub. No.: US 2016/0015932 A1
(43) Pub. Date: Jan. 21, 2016

(54) Peripheral Catheter

(71) Applicant: Janice P. CATUDAL, Marco Island, FL (US)

(72) Inventor: Janice Patricia Catudal, Marco Island, FL (US)

(21) Appl. No.: 14/769,741

(22) PCT Filed: Feb. 27, 2014
(86) PCT No.: PCT/US2014/018853

§ 371 Date: Aug. 21, 2015

Related U.S. Application Data
(60) Provisional application No. 61/770,600, filed on Feb. 28, 2013.

(51) Int. Cl. A61M 25/00; A61M 25/06

(52) U.S. Cl. CPC A61M 25/0097; A61M 25/0606; A61M 25/008

(57) ABSTRACT

Catheter assemblies that can reduce the occurrence of kinking of a catheter when the catheter assembly is secured to a surface of the patient's skin are provided herein. In accordance with various aspects, the catheter hub is shaped such that a catheter extending therefrom lies substantially flat on the patient's skin and/or the distance between the orifice from which the catheter hub extends and the surface of the patient's skin when the catheter assembly is secured thereto is minimized, thereby reducing the stress experienced by the catheter when the catheter hub is secured to the skin.

### RELATED APPLICATIONS

This application claims the benefit of U.S. Provisional Application No. 61/770,600 filed on Feb. 28, 2013, which is incorporated herein by reference in its entirety.

### FIELD

The invention generally relates to catheters, and more particularly, to intravascular (IV) catheters (e.g., short-term indwelling peripheral catheters).

### BACKGROUND

Catheters, particularly peripheral IV catheters, are the most commonly-utilized invasive medical device and are a substantial contributor to healthcare costs. Upon admission to the hospital, peripheral IV catheters are often the first device placed in a patient in order to provide access to the patient's vascular system for the delivery of fluid/nutrients, medication, and/or imaging agents, as well as to provide access for blood sampling and extracorporeal treatment of blood.

Peripheral IV catheters can be placed in various locations of the patient's body depending, for example, on the ease of vascular access. Insertion locations include the hand, wrist, forearm, cubital fossa, and the upper arm. The insertion procedure for an over-the-needle peripheral IV catheter generally includes the following basic steps: (1) a healthcare worker inserts the needle point of a needle extending through the catheter through the skin and into a patient's vein; (2) the catheter, which extends from a catheter hub, is advanced into the vein such that the catheter hub is located near the insertion site; (3) the needle is withdrawn from the catheter while applying digital pressure to the patient's skin proximate to the insertion site to stop the flow of blood through the catheter; and (4) the healthcare worker attaches pre-flushed extension tubing to the catheter hub to seal the catheter and prevent blood backflow. Generally, the catheter hub is then secured to the patient's skin with an adhesive dressing that at least partially covers the insertion site and the catheter hub. The catheter hub can then be coupled to a source of fluid to be administered into the patient's vein, for example.

In accordance with CDC recommendations, most peripheral IV catheters are exchanged every 72-96 hours as a matter of course to purportedly reduce the risk of phlebitis, infiltration, extravasation, occlusion, thrombus formation, and dislodgement. Though a recent study has suggested that it may be preferable to extend the duration of the indwelling catheter until clinically indicated (as opposed to premature scheduled replacement), many catheters must be removed even earlier than the CDC recommended indwelling time due to catheter failure. In the U.S., for example, it is estimated that approximately 50% of peripheral catheterizations fail, thus requiring removal of the catheter and reinsertion of a new catheter at another site. See Rickard CM et al., Routine versus clinically indicated replacement of peripheral intravenous catheters: a randomized controlled equivalence trial, Lancet 380:1066-74 (2012).

It is readily apparent that an indwelling catheter must provide a fluid flow pathway to effectuate the infusion of fluids or to obtain a blood return. Though previous attempts to provide resistance to kinking (e.g., bending or crimping of the catheter that obstructs fluid flow therethrough) have focused, for example, on material modifications to the catheter (see e.g., U.S. Pat. No. 5,993,436), occurrences of kinking of an indwelling catheter nonetheless remains commonplace. In fact, such occurrences are generally left unreported by clinicians as they are often viewed as an inevitable result of catheterization.

Accordingly, there remains a need for improved catheters having a reduced risk of failure.

### SUMMARY

To prevent catheters from having to be removed from an insertion site due to the inability to infuse fluids, for example, the present teachings generally provide catheter assemblies that can reduce the occurrence of kinking of a catheter when the catheter assembly is secured to a surface of the patient's skin. In accordance with various aspects of the present teachings, the catheter extends from the catheter hub such that the catheter can lie substantially flat on the patient's skin relative to known catheter assemblies so as to reduce the stress experienced by the catheter when the catheter hub is secured to the skin.

In accordance with various aspects, certain embodiments of the applicant's teachings relate to an intravascular catheter assembly (e.g., a peripheral IV catheter assembly) that includes a catheter hub extending from a proximal end to a distal nose portion, the catheter hub defining an internal cavity configured to receive at least a portion of a needle assembly. The distal nose portion has an orifice extending therethrough that is configured to receive a needle for insertion into a patient's skin. The peripheral catheter assembly also includes a catheter extending distally from the nose portion to a distal end that is configured to be disposed within a blood vessel, the catheter defining a lumen through which the needle extends when disposed within said orifice. The nose portion also comprises a surface configured to be disposed on the patient's skin when the catheter hub is secured to the patient's skin, the surface being shaped so as to prevent kinking of a length of the catheter extending from the nose portion to an insertion site of the catheter in the patient's skin.

In accordance with various aspects, the surface can be substantially flat. For example, the surface can be a planar surface extending proximally from a terminal distal end of the catheter hub. By way of example, at least a portion of the nose portion can be beveled. In some aspects, the surface can be configured to lie flat against the patient's skin when the catheter hub is secured onto the patient's skin.

In various embodiments, the surface can be shaped as a substantially planar surface extending along at least a portion of the length of the nose portion. In some aspects, the substantially planar surface can extend proximally beyond the nose portion.

In various aspects, the orifice extends along a longitudinal axis, and optionally, the distal nose portion can be asymmetric about this longitudinal axis. In related aspects, the surface can be disposed along the longitudinal axis of the orifice and the surface may be non-parallel or non-perpendicular thereto. For example, in some aspects, the surface can be disposed along the longitudinal axis at an angle between about 5 degrees and about 30 degrees relative to the longitudinal axis (e.g., at an angle between about 8 degrees and about 15 degrees relative to the longitudinal axis). In various aspects, the longitudinal axis of the orifice can be substantially parallel to an insertion angle of the catheter into the patient's skin when the catheter hub is secured onto the patient's skin.

In accordance with various aspects of the present teachings, the surface can be shaped such that the length of the catheter extending from the nose portion to the insertion site of the catheter lies substantially flat against the patient's skin.

In some aspects, catheter assemblies in accordance with the present teachings are configured to minimize the distance between the orifice and the surface of the patient's skin when the catheter assembly is secured thereto. In various aspects, a minimum distance between the orifice and the surface can be less than about 2 mm (e.g., less than about 1 mm)

The catheter can have a variety of configurations but generally provides a lumen through which fluids can be infused into and/or withdrawn from a blood vessel when a distal end of the catheter is disposed therein. In some aspects, the proximal end of the catheter is coupled to the terminal end of the distal nose portion. Alternatively, the proximal end of the catheter can be disposed within (e.g., coaxial with) the catheter hub (e.g., via over-molding), with the catheter extending distally through said orifice. In some aspects, the length of the catheter extending from the nose portion to the insertion site of the catheter is less than about 1 cm when the catheter is secured to the patient's skin.

In various aspects, the peripheral catheter assembly can additionally include a needle assembly removably coupled to the proximal end of the catheter hub, the needle assembly having a needle extending distally through the orifice and the lumen of the catheter. For example, the needle can be configured to be removed from the catheter after placing the catheter (e.g., after disposing the distal end of the catheter within the patient's blood vessel).

In some aspects, a terminal, distal end of the catheter hub can be concave.

In some aspects, the catheter extends from the surface.

These and other features of the applicant's teachings are set forth herein.

### BRIEF DESCRIPTION OF THE DRAWINGS

The person skilled in the art will understand that the drawings, described below, are for illustration purposes only. The drawings are exemplary and are not intended to limit the scope of the teachings in any way.

FIG. 1 depicts a perspective view of an exemplary peripheral catheter assembly in accord with various aspects of the present teachings.

FIG. 2 depicts a side view of the peripheral catheter assembly of FIG. 1.

FIG. 3 depicts a cross-sectional view of another exemplary embodiment of a peripheral catheter assembly.

FIG. 4 depicts a perspective view of another exemplary peripheral catheter assembly in accord with various aspects of the present teachings.

FIG. 5 depicts a top view of another exemplary peripheral catheter assembly in accord with various aspects of the present teachings.

FIG. 6 depicts a side view of another exemplary peripheral catheter assembly in accord with various aspects of the present teachings.

### DETAILED DESCRIPTION

It will be appreciated that for clarity, the following discussion will explicate various aspects of embodiments of the applicant's teachings, while omitting certain specific details wherever convenient or appropriate to do so. For example, discussion of like or analogous features in alternative embodiments may be somewhat abbreviated. Well-known ideas or concepts may also for brevity not be discussed in any great detail. The skilled person will recognize that some embodiments of the applicant's teachings may not require certain of the specifically described details in every implementation, which are set forth herein only to provide a thorough understanding of the embodiments. Similarly it will be apparent that the described embodiments may be susceptible to alteration or variation according to common general knowledge without departing from the scope of the disclosure. The following detailed description of embodiments is not to be regarded as limiting the scope of the applicant's teachings in any manner. As used herein, the terms “about” and “substantially equal” refer to variations in a numerical quantity that can occur, for example, through measuring or handling procedures in the real world; through inadvertent error in these procedures; through differences in the manufacture, source, or purity of compositions or reagents; and the like. Typically, the terms “about” and “substantially equal” as used herein means greater or lesser than the value or range of values stated by 1/10 of the stated values, e.g., ±10%. For instance, a concentration value of about 30% or substantially equal to 30% can mean a concentration between 27% and 33%. The terms also refer to variations that would be recognized by one skilled in the art as being equivalent so long as such variations do not encompass known values practiced by the prior art.

In accordance with various aspects of the applicant's teachings, the catheter assemblies described herein can reduce the occurrence of kinking that frequently results upon insertion and/or during indwelling of an IV catheter, thereby allowing indwelling catheters to remain in place for an extended duration (e.g., until a scheduled replacement or until otherwise clinically indicated). For simplicity, the depicted embodiments are described with reference to a peripheral IV catheter assembly. It should be appreciated, however, that the present teachings are not so limited and catheters used for other types of vascular access such as a central catheters, midline catheters, or peripherally inserted central catheters (PICC) can also be modified in accordance with the present teachings. To prevent catheters from having to be removed from an insertion site due to the inability to infuse fluids caused by kinking of the catheter, for example, catheter hubs in accordance with various aspects of the present teachings can be shaped such that a catheter extending therefrom lies substantially flat on the patient's skin, thereby reducing the stress experienced by the catheter when the catheter hub is secured to the skin. For example, whereas commercially-available catheter assemblies have a flexible catheter that extends from a cylindrical (or truncated conical) distal end of a catheter hub such that the catheter is offset from a surface of the patient's skin as it exits the catheter hub (i.e., the catheter must be bent downward between its exit from the catheter hub to the insertion site), catheter assemblies in accordance with the present teachings can be effective to minimize the distance between the orifice from which the catheter hub extends and the surface of the patient's skin when the catheter assembly is secured thereto. In some aspects, the surface can aid in maintaining the catheter hub in a stable position proximate to the skin by preventing the catheter hub from pivoting about its distal end (e.g., which could elevate the proximal end and make displacement of the catheter more likely, for example, due to increased possibility of snags) and/or subjecting the catheter to increased movement as the catheter hub rocks back and forth on its distal end (e.g., due to movement of the patient), which may lead to increased kinking and/or irritation of the patient's vessel. Importantly, utilizing peripheral catheter assemblies in accordance with various aspects of the present teachings can, for example, reduce kinking which can lead to complete blockage of the fluid flow pathway and/or incorrect dosage per unit time of a medication administered therethrough.

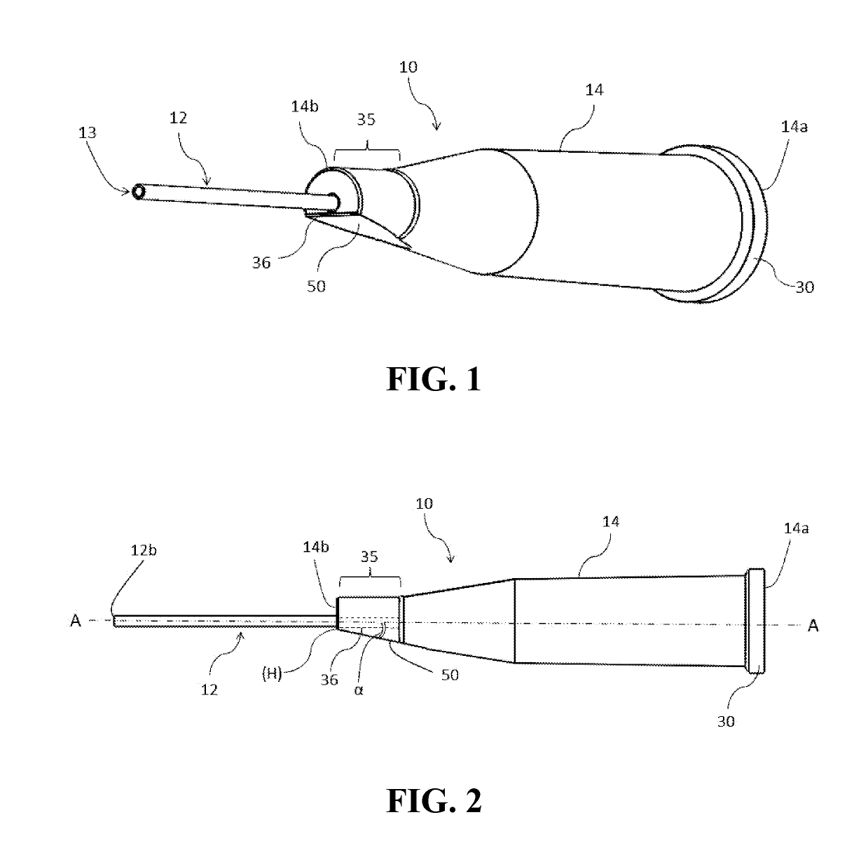

With reference now to FIGS. 1-3, an exemplary peripheral catheter assembly 10 in accordance with various aspects of the applicant's teachings is depicted. As shown in FIG. 1, the catheter assembly 10 generally includes a catheter hub 14 and a catheter 12 extending therefrom. The catheter hub 14 comprises a hollow body that extends from a proximal end 14a (i.e., the end closer to a healthcare worker) to a distal end 14b and defines an interior cavity 24 therein. The catheter 12, which defines a lumen 13, extends from the distal-most end 14b of the catheter hub 14 such that the lumen 13 of the catheter 12 is in fluid communication with the interior cavity 24 of the catheter hub 14.

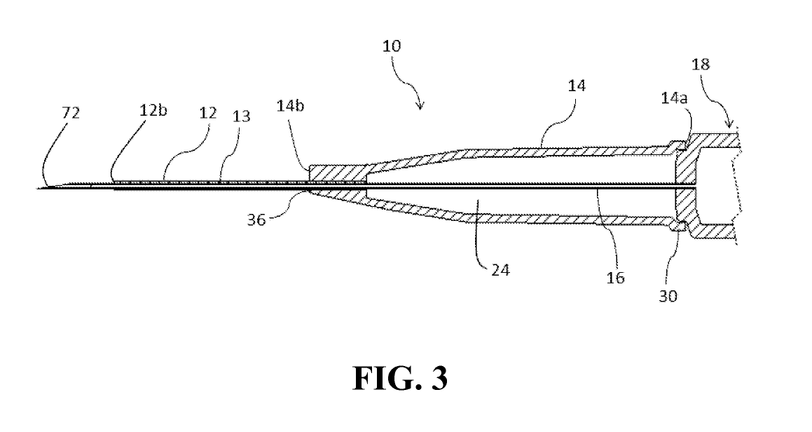

Though the exemplary catheter hub 14 is depicted as being substantially cylindrical, it will be appreciated that the catheter hub 14 can have a variety of shapes in accordance with the present teachings (e.g., square, conical, etc.). Regardless, the distal end 14b of the catheter hub generally comprises a distal nose portion 35 that defines an orifice 36 extending therethrough. The orifice 36, which extends along a longitudinal axis (A), provides a fluid flow pathway between the internal cavity 24 of the catheter hub 14 and a catheter 12 coupled to a terminal distal end 14b of the catheter hub 14, or alternatively, receives therethrough a catheter 12 for providing a fluid flow pathway, as shown in FIG. 3. For example, it will be appreciated that a catheter 12 can be over-molded (e.g., via an injection molding process) such that the proximal end of the catheter 12 is coupled to or disposed within the orifice 36 or within the internal cavity 24 of a catheter hub 14 formed therearound.

The distal nose portion 35 can have a variety of configurations, but is generally shaped so as to prevent the catheter 12 from kinking when the catheter hub 14 is secured to the patient's skin (e.g., via an adhesive dressing). For example, as shown in FIG. 1, though the nose portion 35 is partially cylindrical, the inferior portion of the nose 35 (i.e., a segment of the nose portion 35 disposed below the orifice 36 when the catheter hub 14 is secured to the skin) is beveled such that the distal nose portion 35 comprises a planar surface 50 (e.g., a two-dimensional flat surface) that extends proximally from the distal-most end 14b of the catheter hub 14. In various aspects, the planar surface 50 is configured to lie substantially flat against the skin when the catheter hub 14 is secured to the patient's skin following insertion of the catheter 12 in the patient's vessel. In this manner, the shape of the catheter hub 14, and particularly the nose portion 35, can minimize the distance between the orifice 36 and the surface of the skin when the catheter hub 14 is secured to the patient's skin, thereby decreasing the distance that the catheter 12 must bend between the distal end 14b of the catheter hub 14 towards the insertion site in the patient's skin. Indeed, the catheter 12 can extend from the distal end 14b of the catheter hub 14 such that the catheter 12 lies substantially flat on the skin. For example, as shown in FIG. 2, the minimum distance (H) between the orifice 36 and surface 50 can be less than about 0.5 mm (e.g., less than about 0.2 mm, about 0.1 mm) For example, the wall of the nose portion 35 above the orifice 36 at the distal-most end 14b can be thicker than the wall beneath the orifice 36 so as to protect the catheter 12, for example, while nonetheless maintaining the catheter 12 proximate to skin surface. That is, in accordance with various aspects of the present teachings, the nose portion 35 can be asymmetric about the longitudinal axis (A) of the orifice 36 (i.e., not axisymmetric). Whereas the catheter of conventional catheter assemblies generally extends from the catheter hub such that the catheter is substantially parallel to the surface of the skin when secured thereto, the angled surface 50 can allow the longitudinal axis (A) of the orifice 36 to be made substantially parallel to an insertion angle of the catheter 12 into the patient's skin when the catheter hub 14 is secured to the skin surface so as to reduce kinking of the catheter 12 and/or the length of the catheter 12 disposed above the surface of the skin (i.e., the length of catheter between the distal end 14b and the insertion site). It will be appreciated in light of the present teachings that the surface 50 can be disposed at a variety of angles relative to the longitudinal axis (A) of the orifice 36. By way of non-limiting example, the angle (α) between the longitudinal axis and the axis's projection on the planar surface 50 (i.e., in the plane of the page of FIG. 2) can be between about 0 degrees (e.g., substantially parallel to the longitudinal axis) and about 40 degrees. In some embodiments, for example, the planar surface 50 can be disposed at an angle between about 5 degrees and about 30 degrees relative to the longitudinal axis (e.g., between about 5 degrees and about 15 degrees, about 8 degrees).

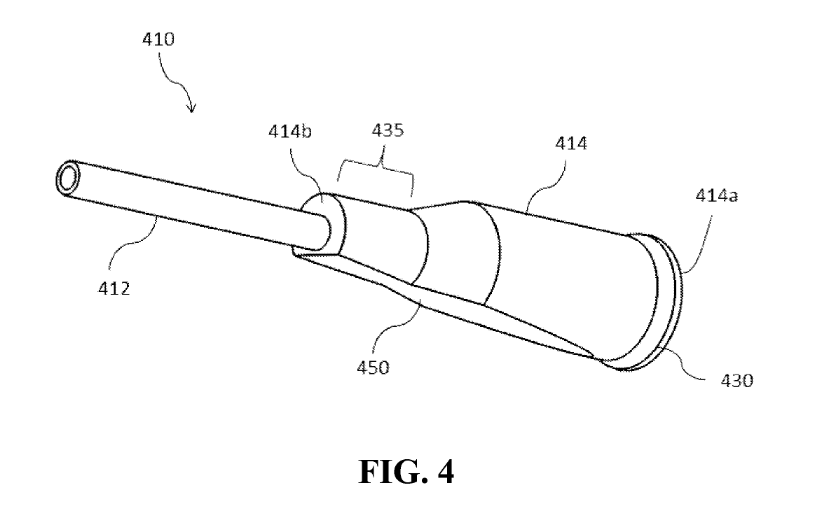

It should be appreciated that the planar surface 50 can also have a variety of sizes and/or lengths. By way of non-limiting example, the planar surface 50 can extend proximally from the distal end 14b of the catheter for a length along the longitudinal axis (A) in a range from about 2 mm to about 20 mm (e.g., about 8 mm, about 1 cm). For example with reference now to FIG. 4, another exemplary peripheral catheter assembly 410 having an exemplary peripheral catheter hub 414 and a catheter 412 extending therefrom in accordance with various aspects of the present teachings is depicted. The catheter hub 414 is substantially similar to the catheter hub 14 of FIGS. 1-3, but differs in that the catheter hub 414 includes a planar surface 450 that extends proximally substantially beyond the distal nose portion 435. Indeed, the planar surface 450 of the catheter hub 414 extends substantially the entire length of the catheter hub 414 and terminates just prior to a connector 430 at the proximal end 414a of the catheter hub 414. It should be appreciated that while the exemplary planar surface 450 depicted in FIG. 4 terminates just prior to the proximal end 414a such that the catheter hub 414 can be coupled, for example, to a conventional needle assembly, it is within the scope of the present teachings that a planar surface 450 in accordance with the present teachings can extend the entire length of the catheter hub 414 (with concomitant modifications to the proximal end 414a or to the needle assembly for coupling thereto).

With reference again to FIG. 3, an exemplary needle assembly 18 is depicted as being coupled to the proximal end 14a of the catheter hub 14 such that a needle 16 extends through the interior cavity 24 of the catheter hub 14 and the lumen 13 of the catheter 12. The needle assembly 18 can have a variety of configurations, but is generally configured to be coupled to the catheter hub 14 such that a sharpened (e.g., beveled) distal tip 72 of the needle 16 extends distally beyond the distal end 12b of the catheter 12. In this manner, the distal tip 72 of the needle can pierce the patient's skin at an insertion site and be advanced into the patient's body such that the catheter 12 is inserted therewith and is also located within the patient's blood vessel. For example, the needle 16 can include a surface feature (e.g., crimp, bump, or clip engagement section) that helps the needle 16 mechanically/frictionally engage the catheter 12 to drive the catheter 12 through the patient's skin as the needle 16 is advanced therethrough. Following insertion, the needle 16 can then be withdrawn while leaving the distal end 12b of the catheter 12 in place. It will be appreciated that the needle assembly 18 can be removably coupled to the catheter hub 14 utilizing a variety of mechanisms known in the art and modified in accordance with the teachings herein. By way of example, the proximal end 14a of the catheter hub 14 can comprise a connector 30 (e.g., a luer-type connector) that can be coupled to a corresponding mating feature on the needle assembly 18, syringe, IV line, etc. In some aspects, for example, the catheter hub 14, catheter 12, and needle assembly 18 can be pre-assembled as a kit such that the healthcare worker can simply remove a sterilized assembly from its packaging, insert the needle tip 72 into the patient, place the catheter 12 which surrounds the needle 16 within the blood vessel, and de-couple the needle assembly 18 from the catheter hub 14. As will be appreciated by a person skilled in the art, the catheter 12 can then be flushed and/or the catheter hub 14 can be sealed or coupled to a source of fluid for infusion, for example.

In accordance with various aspects of the present teachings, the peripheral catheter assembly 10 can be assembled for deployment by coupling the catheter hub 14, catheter 12, and the needle assembly 18 having a needle 16 with a beveled tip 72 such that the beveled tip faces away from the planar surface 50, as shown in FIG. 3 (i.e., the proximal-most portion of the beveled tip 72 is on the opposed side of the longitudinal axis (A) from the planar surface 50). Because insertion of the needle 16 into the skin is generally performed “bevel up,” the planar surface 50 would thus be appropriately positioned to be disposed on the patient's skin surface following insertion of the catheter so as to reduce the risk on kinking. That is, after insertion of the needle 16 and placement of the catheter 12 within the patient's blood vessel, the needle 16 can be removed and the properly oriented catheter hub 14 can be secured to the skin such that the planar surface 50 is in contact therewith.

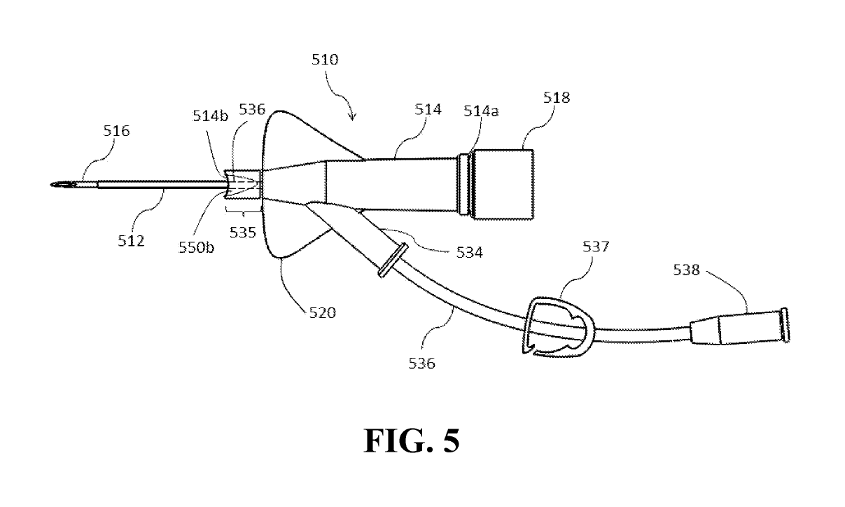

Peripheral catheter assemblies in accordance with the present teachings can also include one or more additional features that can aid in the deployment, securement, and or fluid coupling to the patient's blood vessel, by way of non-limiting examples. With reference now to FIG. 5, another exemplary peripheral catheter assembly 510 is depicted in schematic diagram. Like the catheter assemblies described above, the catheter assembly 510 also includes a catheter hub 514 extending from a proximal end 514a to a distal end 514b, the catheter hub 514 having a distal nose portion 535 that includes a lower planar surface (not shown in this top view), and an upper planar surface 550b (i.e., the nose portion 535 is symmetric relative to at least two planes containing the longitudinal axis (A) of the orifice). As otherwise discussed herein, the lower planar surface of the nose portion 535 can be configured to prevent kinking of the catheter 512. However, whereas the terminal, distal end 14b of the catheter hub 14 depicted in FIG. 1 is perpendicular to the longitudinal axis (A) of the orifice 36, the terminal, distal end 514b of the catheter hub 514 of FIG. 5 instead comprises a concave, curved surface such that the peripheral portions of the distal end 514b extend distally beyond the central portion of the distal end 514b from which the catheter 512 extends (as viewed from the top when disposed on the skin surface). The crescent-shaped distal end 514b can, for example, be effective to increase the stability of the catheter hub 514 when secured to the surface of the skin and/or increase protection of the length of the catheter 512 extending between the distal end 514b and the insertion site.

As shown in FIG. 5, one or more substantially flat wings 520 can additionally extend from the catheter body 514 (e.g., proximal to the nose portion 35) to provide the caregiver with a gripping surface during insertion of the needle 516, as described for example in U.S. Patent Publication No. 20090163871, the teachings of which are incorporated by reference in its entirety. Further, a side port 534 can extend from the catheter body 514 for providing an additional or alternative fluid flow pathway into the internal cavity of the catheter hub 514, also as shown by example in the above-referenced publication. By way of example, the side port 534 can be coupled to a fluid source (e.g., via a length of tubing 536 and fluid coupler 538 (e.g., Luer lock) for delivering fluids to be infused into the internal cavity of the catheter hub 514 when the proximal end 514a of the catheter hub 514 is sealed (e.g., capped) following removal of the needle assembly 518. As shown in FIG. 5, the tubing 536 extending from the side port 534 can include a fluid clamp 537 disposed therearound for controlling the flow of fluid into and/or out of the catheter hub 514.

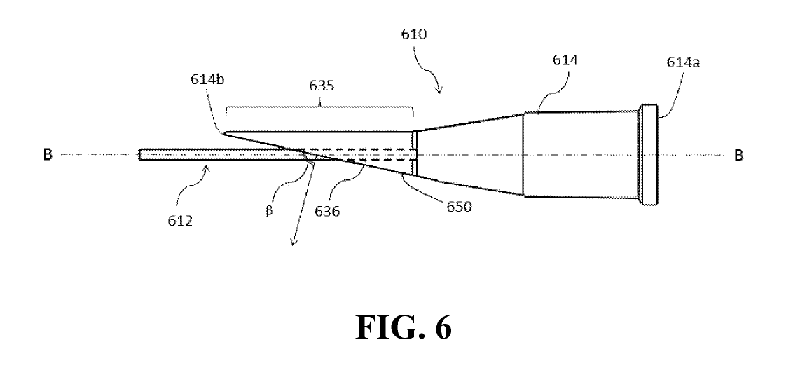

With reference now to FIG. 6, another exemplary peripheral catheter assembly 610 is depicted in schematic diagram. Like the catheter assemblies described above, the catheter assembly 610 also includes a catheter hub 614 extending from a proximal end 614a to a terminal distal end 614b, the catheter hub 614 having a nose portion 635 that includes a planar surface 650 configured to be disposed in contact with a surface of the patient's skin. However, whereas the catheter 12 extends distally from the terminal, distal end 12b of the catheter hub 14 (and the surface 50 extends distally from this end 12b) in the catheter assembly 10 depicted in FIG. 1, the catheter hub 614 differs in that the orifice 636 extends through the planar surface 650 along a longitudinal axis (B), and the catheter extends from this planar surface 650. For example, the terminal, distal end 614b of the catheter hub 614 is distal from where the catheter 612 extends from (or exits) the catheter hub 614. The catheter 612 can extend from the surface 650 at a variety of angles for insertion into the patient when the surface 650 is disposed in contact with the skin. By way of non-limiting example, the catheter 612 can extend away from the flat surface (e.g., toward the distal end 614b) at an angle (β) between about 60 degrees and about 85 degrees relative to the normal of the surface 650 (e.g., between about 85 degrees and about 75 degrees, about 82 degrees).

In various aspects, the planar surface 650 can be configured to lie substantially flat against the skin when the catheter hub 614 is secured to the patient's skin following insertion of the catheter 612 in the patient's vessel. In this manner, the shape of the catheter hub 614, and particularly the flat surface 650 from which the catheter 612 extends, enables the catheter 612 to enter the patient's skin at an insertion site directly adjacent (e.g., under) the flat surface 650 such that the length of the catheter 612 disposed above the surface of the skin (i.e., the length of catheter between the distal end 614b and the insertion site) is minimized It will further be appreciated that the catheter hub 614 can therefore serve to protect the catheter 612 by having the nose portion 635 (e.g., the flat surface 650) fully surround the catheter 612 when the surface 650 is disposed in contact with the patient's skin.

The peripheral catheter assemblies described herein can be manufactured using a variety of materials and techniques. By way of non-limiting example, the catheter hub can be formed from a rigid polymeric material while the catheter comprises a more flexible polymeric material such as vyalon, polyurethane, or another soft plastic. In some aspects, the catheter hub can be injection molded in net shape or near net shape and the molding process can comprise insert molding or over-molding in conjunction with other components such as the catheter. The catheter hub manufacturing process can also include post-molding operations such as surface finishing or removal of sharp edges and mold flash. By way of example, the flat surface 50 as described above with reference to FIGS. 1-3, for example, can be molded in as a net shape, or alternatively, can be produced by a post-molding operation using techniques such as machining, laser cutting, or grinding.

The section headings used herein are for organizational purposes only and are not to be construed as limiting. While the applicant's teachings are described in conjunction with various embodiments, it is not intended that the applicant's teachings be limited to such embodiments. On the contrary, the applicant's teachings encompass various alternatives, modifications, and equivalents, as will be appreciated by those of skill in the art.

What is claimed is:

1. An intravascular catheter assembly, comprising:
a catheter hub extending from a proximal end to a distal nose portion and defining an internal cavity configured to receive at least a portion of a needle assembly, the distal nose portion having an orifice extending therethrough and configured to receive a needle for insertion into a patient's skin; and
a catheter extending distally from the nose portion to a distal end configured to be disposed within a patient's blood vessel, the catheter defining a lumen through which the needle extends when disposed within said orifice,
wherein the nose portion comprises a surface configured to be disposed on the patient's skin when the catheter hub is secured to the patient's skin, the surface being shaped so as to prevent kinking of a length of the catheter extending from the nose portion to an insertion site of the catheter in the patient's skin.

2. The intravascular catheter assembly of claim 1, wherein said surface comprises a planar surface extending proximally from a terminal distal end of the catheter hub.

3. The intravascular catheter assembly of claim 1, wherein the surface is configured to lie flat against the patient's skin when the catheter hub is secured onto the patient's skin.

4. The intravascular catheter assembly of claim 1, wherein said orifice extends along a longitudinal axis.

5. The intravascular catheter assembly of claim 4, wherein said distal nose portion is asymmetric about said longitudinal axis.

6. The intravascular catheter assembly of claim 4, wherein the surface is disposed along the longitudinal axis of the orifice and the surface is not parallel or perpendicular thereto.

7. The intravascular catheter assembly of claim 4, wherein said surface is disposed along the longitudinal axis at an angle between about 5 degrees and about 30 degrees relative to the longitudinal axis.

8. The intravascular catheter assembly of claim 7, wherein said angle is between about 8 degrees and about 15 degrees relative to the longitudinal axis.

9. The intravascular catheter assembly of claim 4, wherein the longitudinal axis of the orifice is substantially parallel to an insertion angle of the catheter into the patient's skin when the catheter hub is secured onto the patient's skin.

10. The intravascular catheter assembly of claim 1, wherein said surface comprises a substantially planar surface extending along at least a portion of the length of the nose portion.

11. The intravascular catheter assembly of claim 10, wherein said substantially planar surface extends proximally beyond the nose portion.

12. The intravascular catheter assembly of claim 1, wherein said surface is shaped such that the length of the catheter extending from the nose portion to the insertion site of the catheter lies substantially flat against the patient's skin.

13. The intravascular catheter assembly of claim 1, wherein a minimum distance between the orifice and the surface is less than about 2 mm.

14. The intravascular catheter assembly of claim 1, wherein a minimum distance between the orifice and the surface is less than about 1 mm.

15. The intravascular catheter assembly of claim 1, wherein a proximal end of the catheter is coupled to the distal nose portion.

16. The intravascular catheter assembly of claim 1, wherein a proximal end of the catheter is disposed within the catheter hub, said catheter extending distally through said orifice.

17. The intravascular catheter assembly of claim 1, wherein the length of the catheter extending from the nose portion to the insertion site of the catheter is less than about 1 cm when the catheter is secured to the patient's skin.

18. The intravascular catheter assembly of claim 1, further comprising a needle assembly coupled to the proximal end of the catheter hub, the needle assembly having a needle extending distally through said orifice and catheter.

19. The intravascular catheter assembly of claim 18, wherein said needle is configured to be removed from the catheter after disposing said distal end of the catheter within the patient's blood vessel.

20. The intravascular catheter assembly of claim 1, wherein a terminal, distal end of the catheter hub is concave.

21. The intravascular catheter assembly of claim 1, wherein the catheter extends from said surface.
